(19) United States
(12) Patent Application Publication
(10) Pub. No.: US 2014/0163095 A1
(43) Pub. Date: Jun. 12, 2014

(54) USE OF ERIBULIN IN THE TREATMENT OF BREAST CANCER

(71) Applicant: Eisai R&D Management Co., Ltd., Tokyo (JP)

(72) Inventors: Alton KREMER, Weston, CT (US); Peter Tarassoff, Mount Arlington, NJ (US); Claudio Savulsky, St. Albans (GB); Martin Olivo, Westwood, NJ (US); Yi He, Hillsborough, NJ (US); D. Matthew Guo, Montvale, NJ (US)

(73) Assignee: Eisai R&D Management Co., Ltd., Tokyo (JP)

(21) Appl. No.: 14/096,827

(22) Filed: Dec. 4, 2013

Related U.S. Application Data
(60) Provisional application No. 61/878,204, filed on Sep. 16, 2013.
(60) Provisional application No. 61/733,238, filed on Dec. 4, 2012.

(51) Int. Cl. A61K 31/357

(52) U.S. Cl. CPC A61K 31/357
USPC 514/450

(57) ABSTRACT

The invention provides methods of treating breast cancer in subjects having locally advanced or metastatic breast cancer.

Cancer is a term used to describe a wide variety of diseases that are each characterized by the uncontrolled growth of a particular type of cell. It begins in a tissue containing such a cell and, if the cancer has not spread to any additional tissues at the time of diagnosis, may be treated by, for example, surgery, radiation, or another type of localized therapy. However, when there is evidence that cancer has metastasized from its tissue of origin, different approaches to treatment are typically used. Indeed, because it is not possible to determine the extent of metastasis, systemic approaches to therapy are usually undertaken when any evidence of spread is detected. These approaches involve the administration of, for example, chemotherapeutic drugs that interfere with the growth of rapidly dividing cells, such as cancer cells.

Halichondrin B is a structurally complex, macrocyclic compound that was originally isolated from the marine sponge Halichondria okadai, and subsequently was found in Axinella sp., Phakellia carteri, and Lissodendoryx sp. A total synthesis of halichondrin B was published in 1992 (Aicher et al., J. Am. Chem. Soc. 114:3162-3164, 1992). Halichondrin B has been shown to inhibit tubulin polymerization, microtubule assembly, beta^{S}-tubulin crosslinking, GTP and vinblastine binding to tubulin, and tubulin-dependent GTP hydrolysis in vitro. This molecule has also been shown to have anti-cancer properties in vitro and in vivo. Halichondrin B analogs having anti-cancer activities are described in U.S. Pat. No. 6,214,865 B1.

Eribulin is a synthetic analog of halichondrin B. Eribulin is also known as ER−086526, and has been assigned CAS number 253128-41-5 and US NCI designation number NSC-707389. The mesylate salt of eribulin (eribulin mesylate, which is marketed under the trade name HALAVEN® and is also known as E7389) is approved for the treatment of patients with breast cancer who have previously received at least two chemotherapeutic regimens for the treatment of metastatic disease that should have included an anthracycline and a taxane in either the adjuvant or metastatic setting.

The chemical name for eribulin mesylate is 11,15:18,21:24,28-Triepoxy-7,9-ethano-12,15-methano-9H,15H-furo[3,2-i]furo[2′,3′:5,6]pyrano[4,3-b][1,4]dioxacyclopentacosin-5(4H)-one, 2-[(2S)-3-amino-2-hydroxypropyl]hexacosahydro-3-methoxy-26-methyl-20,27-bis(methylene)-, (2R,3R,3aS,7R,8aS,9S,10aR,11S,12R,13aR,13bS,15S,18S,21S,24S,26R,28R,29aS)-methanesulfonate (salt), and it can be depicted as

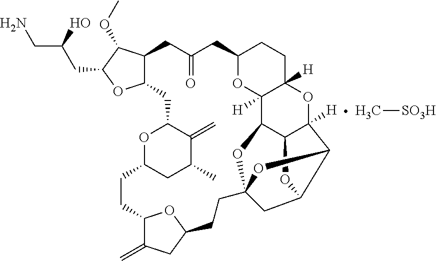

### SUMMARY OF THE INVENTION

The invention provides methods of treating breast cancer in a subject (e.g., a human subject, such as a human breast cancer patient) selected as having (i) HER2-negative, (ii) estrogen receptor (ER)-negative, or (iii) HER2-negative, ER-negative, and progesterone receptor (PR)-negative (triple negative) breast cancer. The methods involve administering to the subject eribulin or a pharmaceutically acceptable salt thereof (e.g., eribulin mesylate). The eribulin or pharmaceutically acceptable salt thereof can be administered, for example, for 2-5 minutes intravenously on days 1 and 8 of a 21 day cycle, optionally at a dose of 1.4 mg/m².

In some embodiments, the subject has locally advanced or metastatic breast cancer. In further embodiments, the subject has undergone zero, one, or two prior breast cancer treatment regimens, for example, breast cancer treatment regimen(s) including chemotherapy or biologic therapy. In various examples, the subject has undergone a prior breast cancer treatment regimen involving administration of one or more of an antibody (e.g., trastuzumab), a hormonal agent, capecitabine, an anthracycline (e.g., doxorubicin, epirubicin, daunorubicin, or idarubicin), and a taxane (e.g., paclitaxel or docetaxel). In other embodiments, the subject has not previously been treated with an anthracycline or a taxane.

The methods of the invention can also include a step of selecting a subject having (i) HER2-negative, (ii) estrogen receptor (ER)-negative, or (iii) HER2-negative, ER-negative, and progesterone receptor (PR)-negative (triple negative) breast cancer for treatment as described herein, and optionally also testing the subject for HER2, ER, and/or PR status.

The methods of the invention may further include: (a) selection of eribulin or a pharmaceutically acceptable salt thereof to treat said subject, instead of capecitabine, or (b) increasing 1-year overall survival by treatment of said subject with eribulin or a pharmaceutically acceptable salt thereof, relative to capecitabine, based on detection of the breast cancer of said subject as being (i) HER2-negative, (ii) estrogen receptor (ER)-negative, or (iii) HER2-negative, ER-negative, and progesterone receptor (PR)-negative (triple negative).

Further, the invention provides methods of identifying a breast cancer patient as a candidate for treatment with eribulin or a pharmaceutically acceptable salt thereof (e.g., eribulin mesylate). These methods include assessing the status of HER2, ER, and/or PR of the breast cancer of the patient. Determining that the patient is (i) HER2-negative, (ii) ER-negative, or (iii) HER2-negative, ER-negative, and PR-negative (triple negative) identifies the patient as a candidate for treatment with eribulin or a pharmaceutically acceptable salt thereof (e.g., eribulin mesylate). These methods can further include obtaining and analyzing a breast cancer tissue sample from the patient and/or administering eribulin or a pharmaceutically acceptable salt thereof (e.g., eribulin mesylate) to the patient. These methods can also include obtaining and analyzing a breast cancer sample from the patient, and/or administration of eribulin or a pharmaceutically acceptable salt thereof (e.g., eribulin mesylate) to the patient.

The invention also includes methods of selecting treatment for a breast cancer patient, including assessing the status of HER2, ER, and/or PR of the breast cancer of the patient, wherein determining that the patient is (i) HER2-negative, (ii) ER-negative, or (iii) HER2-negative, ER-negative, and PR-negative (triple negative) indicates selection of eribulin or a pharmaceutically acceptable salt thereof for treatment of the patient. These methods can also include obtaining and analyzing a breast cancer sample from the patient, and/or administration of eribulin or a pharmaceutically acceptable salt thereof (e.g., eribulin mesylate) to the patient.

Also included in the invention is eribulin or a pharmaceutically acceptable salt thereof (e.g., eribulin mesylate) for use in the treatment of breast cancer in a subject having (i) HER2-negative, (ii) estrogen receptor (ER)-negative, or (iii) HER2-negative, ER-negative, and progesterone receptor (PR)-negative (triple negative) breast cancer, as described herein. The subject can be as described above and elsewhere herein, and can be treated as described herein.

Further, the invention provides an in vitro method for assessing the suitability of a subject such as a breast cancer patient to treatment with eribulin or a pharmaceutically acceptable salt thereof, characterized in that the status of HER2, ER, and/or PR in a sample taken from the subject is measured and wherein determination of the sample as being (i) HER2-negative, (ii) ER-negative, or (iii) HER2-negative, ER-negative, and PR-negative (triple negative) is indicative of a subject suitable for treatment with eribulin or a pharmaceutically acceptable salt thereof. The subject can be as described above and elsewhere herein, and can be treated as described herein.

The invention also provides use of an in vitro method of assessing the status of HER2, ER, and/or PR of in a sample taken from a subject such as a breast cancer patient to establish the suitability of the subject for treatment with eribulin or a pharmaceutically acceptable salt thereof, wherein determining of the sample as being (i) HER2-negative, (ii) ER-negative, or (iii) HER2-negative, ER-negative, and PR-negative (triple negative) is indicative of a subject suitable for treatment with eribulin or a pharmaceutically acceptable salt thereof. The subject can be as described above and elsewhere herein, and can be treated as described herein.

Other features of the invention are described below and shown in the drawings.

### BRIEF DESCRIPTION OF THE DRAWINGS

FIG. 1 is a schematic illustration of the design and parameters of a phase III clinical trial comparing eribulin mesylate and capecitabine.

FIG. 2 is a graph showing the overall survival of patients treated with eribulin mesylate or capecitabine.

FIG. 3 is a graph showing a Kaplan-Meier plot of progression-free survival of patients in the clinical trial.

FIG. 4 is a graph showing metastasis-free survival of patients treated with eribulin mesylate or capecitabine.

FIG. 5 is a graph showing time to new metastasis observed in the central nervous system, lungs, or liver for patients treated with eribulin mesylate or capecitabine.

FIG. 6 is a forest plot showing overall survival of patients based on the receptor status of their breast cancer.

FIG. 7A is a graph showing a statistically significant increase in median survival in patients with breast cancers characterized as triple negative and treated with eribulin or as compared to patients treated with capecitabine. FIG. 7B is a graph showing that median survival in patients with breast cancers that are not characterized as triple negative is similar in patients treated with eribulin as compared to patients treated with capecitabine.

FIG. 8 is a schematic illustration of the design and parameters of a phase II clinical trial for use of eribulin mesylate as a first-line therapy for locally recurrent or metastatic HER2 negative breast cancer.

FIGS. 9 and 11 are graphs showing a Kaplan-Meier plot of progression-free survival of patients administered eribulin mesylate as a first-line therapy for locally recurrent or metastatic HER2 negative breast cancer.

FIGS. 10 and 12 are waterfall graphs showing the percentage change in total sum of target lesion diameters from baseline to post-baseline nadir (RECIST 1.1).

### DESCRIPTION OF THE INVENTION

The invention is based, at least in part, on the observation that certain breast cancer patients benefit more from treatment with eribulin mesylate as compared to treatment by a current standard of care drug, capecitabine. More specifically, the invention provides methods of treating breast cancer (such as locally advanced or metastatic breast cancer) in patients selected as having breast cancer with one of the following receptor characteristics: (i) HER2 (human epidermal growth factor receptor 2; ERBB2) negative (HER2−), (ii) estrogen receptor negative (ER−), or (iii) HER2−, ER−, and progesterone receptor negative (PR−) (i.e., triple negative). The methods of the invention involve administration of a therapeutically effective amount of eribulin or a pharmaceutically acceptable salt thereof (e.g., eribulin mesylate) to such patients.

Eribulin and pharmaceutically acceptable salts of eribulin (such as the mesylate salt of eribulin, which is marketed under the trade name HALAVEN® and is also known as E7389; see structure set forth above) can be made using methods described, for example, in U.S. Pat. No. 6,214,865; U.S. Pat. No. 8,093,410; U.S. Pat. No. 8,203,010; U.S. Patent Application Publication No. 2007-0244187-A1; U.S. Patent Application Publication No. 2011-054194-A1; and Kim et al., J. Am. Chem. Soc. 131 (43):15636-15641, 2009, the contents of each of which are incorporated herein by reference.

The phrase “pharmaceutically acceptable salt,” as used herein with respect to eribulin, is a salt formed from an acid and a basic nitrogen group of eribulin. Examples of such salts include acid addition salts and base addition salts, such as inorganic acid salts or organic acid salts (e.g., hydrochloric acid salt, sulfuric acid salt, citrate, hydrobromic acid salt, hydroiodic acid salt, nitric acid salt, bisulfate, phosphoric acid salt, super phosphoric acid salt, isonicotinic acid salt, acetic acid salt, lactic acid salt, salicylic acid salt, tartaric acid salt, pantothenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt (i.e., mesylate salt), ethanesulfonic acid salt, benzenesulfonic acid salt, p-toluenesulfonic acid salt, pamoic acid salt (pamoate)), as well as salts of aluminum, calcium, lithium, magnesium, calcium, sodium, zinc, and diethanolamine.

### Dosage and Administration

Treatment regimens involving administration of therapeutically effective amounts of drugs such as eribulin (or pharmaceutically acceptable salts thereof, such as eribulin mesylate) are typically designed on the basis of at least one of the following parameters and, more typically, on the basis of many or all of the parameters: dosage, formulation, route of administration, and/or frequency of administration. Selection of particular parameters of a treatment regimen can be based on known treatment parameters for eribulin previously established in the art, such as those described in the Dosage and Administration protocols set forth in the FDA Approved Label for HALAVEN®, the entire contents of which are incorporated herein by reference. For example, eribulin mesylate can be administered for 2-5 minutes intravenously on days 1 and 8 of a 21 day cycle, at, for example, a dose of 1.4 mg/m². Alternatively, if a dose reduction is indicated (e.g., due to hepatic or renal impairment), the drug can be administered at a dose of 0.7 mg/m² or 1.1 mg/m². Various modifications to dosage, formulation, route of administration, and/or frequency of administration can be made based on various factors including, for example, the disease, age, sex, and weight of the patient, as well as the severity or stage of cancer, and the patient's response (see, for example, U.S. Pat. No. 6,653,341 and U.S. Pat. No. 6,469,182, the entire contents of each of which are hereby incorporated herein by reference). In addition, multiple cycles can be administered (e.g., 4-8, 5-7, or 6 cycles), as determined to be appropriate by one skilled in the art.

For administration to a patient, eribulin or a pharmaceutically acceptable salt thereof, such as eribulin mesylate, typically is formulated into a pharmaceutical composition including the drug and a pharmaceutically acceptable carrier or diluent (e.g., 0.9% Sodium Chloride Injection, USP). Therapeutic compositions typically are sterile and adequately stable under the conditions of manufacture and storage.

The methods of the invention can be carried out in conjunction with the administration of supportive agents such as antiemetics, which are drugs that are used to reduce the nausea and vomiting that are common side effects of cancer chemotherapy. Examples of such drugs include major tranquilizers (e.g., phenothiazines, such as chlorpromazine and prochlorperazine), dopamine antagonists (e.g., metoclopramide), serotonin antagonists (e.g., ondansetron and granisetron), cannabinoids (e.g., dronabinol), and benzodiazepine sedatives. An additional example of a supportive drug that can be administered in conjunction with the methods of the invention is erythropoietin.

As used herein, a “therapeutically effective amount” of eribulin or a pharmaceutically acceptable salt thereof (e.g., eribulin mesylate) means an amount of the drug that is capable of treating breast cancer. The dose of a drug to be administered according to the invention will, of course, be determined in light of the particular circumstances surrounding the case including, for example, the drug administered, the route of administration, the condition of the patient, and the nature of the pathological condition being treated, for example, the stage of breast cancer.

As used herein, “pharmaceutically acceptable carrier or diluent” includes any and all solvents, dispersion media, coatings, antibacterial and antifungal agents, isotonic and absorption delaying agents, and the like that are physiologically compatible. The carrier or diluent can be suitable for parenteral (e.g., intravenous, intramuscular, subcutaneous, or intrathecal) administration (e.g., by injection or infusion). A specific example is 0.9% Sodium Chloride Injection, USP.

As used herein, the term “subject” or “patient” refers to human and non-human animals, e.g., veterinary patients. The term “non-human animal” includes vertebrates, e.g., mammals, such as non-human primates, mice, rabbits, sheep, dogs, cats, horses, cows, or other rodent, ovine, canine, feline, equine, or bovine species. In one embodiment, the subject is a human.

### Patient Selection

As noted above, the methods of the invention are used in the treatment of breast cancer, such as locally advanced or metastatic breast cancer, in particular patient populations.

As used herein, the term “breast cancer” refers generally to the uncontrolled growth of breast tissue and, more specifically, to a condition characterized by anomalous rapid proliferation of abnormal cells in one or both breasts of a subject. The abnormal cells often are referred to as malignant or “neoplastic” cells, which are transformed cells that can form a solid tumor. The term “tumor” refers to an abnormal mass or population of cells (i.e., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize. In breast cancer, neoplastic cells may be identified in one or both breasts only and not in another tissue or organ, in one or both breasts and one or more adjacent tissues or organs (e.g., lymph node), or in a breast and one or more non-adjacent tissues or organs to which the breast cancer cells have metastasized.

The breast cancer can be, for example, adenocarcinoma, inflammatory breast cancer, recurrent (e.g., locally recurrent), locally advanced, and/or metastatic breast cancer. In some embodiments, the breast cancer is endocrine refractory or hormone refractory. The terms “endocrine refractory” and “hormone refractory” refer to a cancer that is resistant to treatment with hormone therapy for breast cancer, e.g., aromatase inhibitors or tamoxifen. Breast cancers arise most commonly in the lining of the milk ducts of the breast (ductal carcinoma) or in the lobules where breast milk is produced (lobular carcinoma). Accordingly, in various embodiments of the invention, the breast cancer can be ductal carcinoma or lobular carcinoma. Cancerous cells from the breast(s) may invade or metastasize to any other organ or tissue of the body. For example, breast cancer cells often invade lymph node cells and/or metastasize to the liver, brain, and/or bone.

In various embodiments of the present invention, the patient may be suffering from Stage I, Stage II, Stage III, or Stage IV breast cancer. The stage of the breast cancer of a patient can be classified based on features such as tumor size, lymph node involvement, and the extent of metastasis, as is well known in the art.

The methods of the invention can be carried out, for example, with breast cancer patients who have had no or only a limited amount of prior treatment (for example, one, two, or three treatment regimens involving chemotherapy and/or biological therapy) and in patients with locally advanced or metastatic breast cancer, preferably no more than two prior regimens.

In the case of patients selected on the basis of prior treatment, the methods of the invention include the treatment of patients who have not received any prior treatment regimen (for example, a treatment regimen involving chemotherapy and/or biological therapy). In these patients, treatment according to the methods of the invention can be called, in various examples, a “first line” treatment.

In some embodiments, the methods of the invention may be used with patients who have received a single prior regimen of treatment (for example, treatment involving chemotherapy and/or biological therapy), in which case treatment according to the methods of the invention can be called, in various examples, a “second line” treatment. These patients typically have been treated previously with a single regimen involving administration of, for example, an antibody (e.g., trastuzumab), a hormonal agent, capecitabine, an anthracycline (e.g., doxorubicin, epirubicin, daunorubicin, or idarubicin), a taxane (e.g., paclitaxel or docetaxel), a platinum (e.g., cisplatin, or carboplatin), or a combination thereof. In other embodiments, the methods of the invention may be used with patients that have had no more than two prior treatment regimens. In other embodiments, the methods of the invention may be used with patients that have had two or more prior treatment regimens (and can be called, in various examples, “third line”). In some embodiments, the prior regimens have included an anthracycline, a taxane, or both. In some embodiments, patients with known HER2/neu overexpressing tumors may have been treated with trastuzumab. In other embodiments patients with known estrogen and/or progesterone receptor positive disease may have been treated with hormonal therapy.

As is understood in the art, a treatment regimen in cancer therapy does not typically involve administration of a single dose of a drug. Rather, a treatment regimen involves multiple cycles of drug administration that are typically designed so that a patient has the opportunity to recover from side effects of the drug between the cycles. Thus, for example, a patient who has received a single prior treatment regimen of a drug may have received the drug, for example, in 3-8 different doses separated from one another by 1-2 weeks. Such an administration regimen, or a substantial portion thereof (e.g., at least half of the regimen), can be considered as a single prior treatment regimen in the selection of patients to treat with eribulin (or a pharmaceutically acceptable salt thereof, such as eribulin mesylate) as a second-line treatment, according to the methods of the invention.

Breast cancer cells in patient samples can be characterized by the presence or absence of estrogen receptors (ER), progesterone receptors (PR), and/or human epidermal growth factor receptor 2 (HER2). Assessment of ER, PR, and HER2 status can be done using standard methods and kits that are well known in the art (see, e.g., Hammond et al., J. Clin. Oncol. 28(16):2784-2795, 2010; Wolff et al., J. Clin. Oncol. 31(31):3997-4014, 2013; and references cited therein; also see tests available from Quest Diagnostics (questdiagnostics.com)). For example, HER2, ER, and PR status can be determined by immunohistochemistry (IHC). Furthermore, HER2 status can be determined by detection of gene amplification by, for example, in situ hybridization (ISH, e.g., fluorescence in situ hybridization (FISH)) analysis of a breast cancer tissue biopsy according to, for example, National Comprehensive Cancer Network [NCCN] guidelines. In carrying out these standard methods, those of skill in the art can readily determine whether a breast cancer tissue sample is HER2−, ER−, and/or HER2−, thus qualifying the patient from whom the sample is obtained for selection for treatment according to the methods of the invention, as described herein.

As an example, in the case of ER and PR testing, various accepted assays in the field utilize a cut-off of <1% positive tumor nuclei in a sample as tested by IHC utilizing anti-ER or anti-PR antibodies, or an Allred score of <3, in the presence of expected reactivity of controls, in order to identify negative samples (see, e.g., Hammond et al., supra, and Quest Diagnostics assays). In the case of HER2 status, as an example, various accepted assays utilize evidence of protein overexpression (IHC; IHC 1+ or IHC 0 indicates negative) or gene amplification (HER2 copy number (single probe; HER2 copy number <4.0 signals/cell indicates negative) or HER2/CEP17 ratio (dual probe; HER2 copy number <4.0 signals/cell, and HER2/CEP17 ratio <2.0, indicates negative)) by in situ hybridization (ISH) (see, e.g., Wolff et al., supra, and Quest Diagnostics assays). Examples of Quest Diagnostics assays that can be used include the following: ER/PR, Paraffin Block; ER/PR/HER2 with Reflex to HER2 FISH, Paraffin Block; Estrogen Receptor (ER), IHC; HER−2, IHC; HER2 (HercepTest (R), IHC; and HER−2, IHC with Reflex to HER−2, FISH.

The invention also includes methods of identifying breast cancer patients as candidates for treatment with eribulin or a pharmaceutically acceptable salt thereof (e.g., eribulin mesylate), as well as methods of selecting a treatment for a breast cancer patient. Further, the invention also includes methods of optimizing therapy for a breast cancer patient using these approaches, and determining whether a patient is likely to respond to such treatment, as well as methods of selecting a patient for treatment with eribulin (or a pharmaceutically acceptable salt thereof, such as eribulin mesylate), versus capecitabine. The invention also includes methods of increasing overall survival (e.g., 1-year OS) in patients by treatment with eribulin or a pharmaceutically salt thereof (e.g., eribulin mesylate) as described herein, relative to treatment with capecitabine. These methods involve assessing the status of HER2, ER, and/or PR of the breast cancer of a patient as described herein. Determining that the patient is (i) HER2-negative, (ii) ER-negative, or (iii) HER2-negative, ER-negative, and PR-negative (i.e., triple negative) identifies the patient as a candidate for selection for treatment with eribulin or a pharmaceutically acceptable salt thereof (e.g., eribulin mesylate), or indicates selection of such treatment for a patient (e.g., versus capecitabine). These methods can optionally also include obtaining a cancer tissue biopsy from the patient and/or administering eribulin or a pharmaceutically acceptable salt thereof (e.g., eribulin mesylate) to the patient, as described herein.

### EXAMPLES

### Example 1

### Clinical Studies of Eribulin: Phase III Clinical Trial Comparing the Efficacy of Eribulin to the Efficacy of the Standard of Care Drug Capecitabine for Treatment of Breast Cancer

A global, randomized, open-label, two-parallel-arm, phase III clinical trial of eribulin (eribulin mesylate) and capecitabine was performed. Capecitabine is widely used in the treatment of metastatic breast cancer in 1^{st}-, 2^{nd}-, and 3^{rd}-line settings. Eribulin mesylate is approved for treating patients who have previously received at least two chemotherapeutic regimens for the treatment of metastatic breast cancer, where the prior therapy should have included an anthracycline and a taxane. This study demonstrates that treatment of breast cancer with eribulin compares favorably with treatment with capecitabine in 1^{st}, 2^{nd}, and 3^{rd} line regimens and with certain patient populations, treatment with eribulin provides superior results.

This study randomized 1102 patients who had up to three prior chemotherapy regimens, and no more than two prior regimens for advanced and/or metastatic disease. The prior regimens must have included an anthracycline and a taxane, either in the (neo) adjuvant setting or for locally advanced or metastatic disease. Patients must have had documented evidence of progression during or after their most recent anti-cancer therapy. In addition, patients with known HER2/neu overexpressing tumors may have been treated with trastuzumab in centers where this treatment is available, and patients with known estrogen and/or progesterone receptor positive disease may have been treated with hormonal therapy. Patients were randomized to receive either eribulin mesylate as an intravenous (IV) infusion of 1.4 mg/m² over 2-5 minutes on days 1 and 8 every 21 days or capecitabine as an oral administration of 2.5 g/m²/day administered twice daily in two equal doses on days 1 to 14 every 21 days.

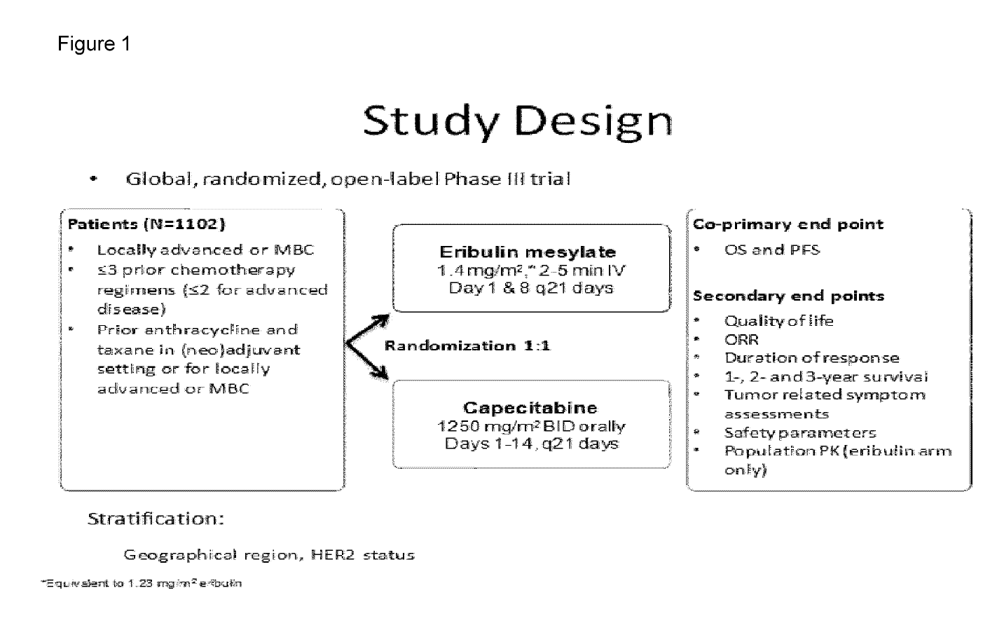

This study was designed to have co-primary endpoints, overall survival (OS) and progression free survival (PFS), with alpha spending 0.04 and 0.01, respectively. The study design, parameters, and secondary endpoints are summarized in FIG. 1.

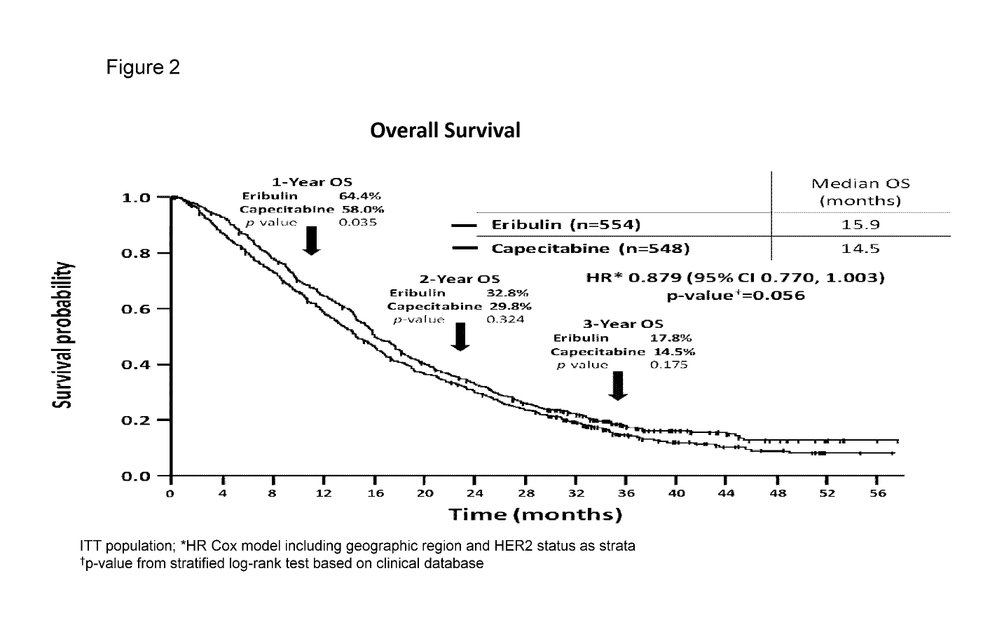

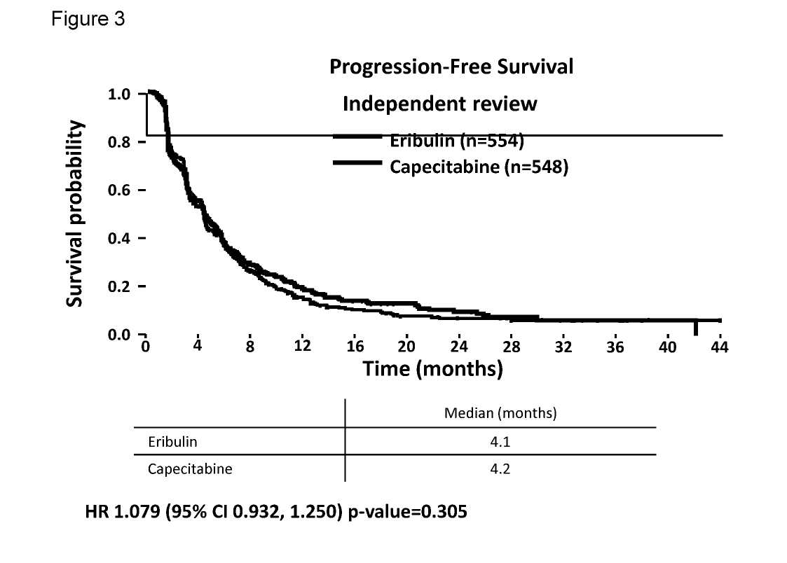

The trial established that eribulin is as effective as capecitabine in overall patient survival (FIG. 2). It is noteworthy that eribulin confers a statistically significant improvement in 1-year OS relative to capecitabine. In particular, a trend favoring improved overall survival with eribulin emerged early and was maintained throughout the study (median 15.9 versus 14.5 months (hazard ratio [HR] 0.88; 95% confidence interval [CI] 0.77, 1.00; p =0.056). However, progression-free survival was not significantly different between erubilin and capecitabine (median 4.1 versus 4.2 months (HR 1.08; 95% CI 0.93, 1.25; P=0.30); FIG. 3). These data demonstrate that the treatment effects of eribulin were comparable to that of capecitabine in the 1^{st}-, 2^{nd}-, and 3^{rd}-line settings.

We next investigated the apparent discordance between OS and PFS in this study by carrying out a post-hoc analysis to assess the relationship between OS and different events defining disease progression. Progression events (disease progression determined by Response Evaluation Criteria in Solid Tumors [RECIST, version 1.0]) were categorized as: (i) emergence of a new lesion/metastasis, defined here as a lesion identified at progression that had not been previously reported, (ii) increase in size of an existing (previously reported) lesion (target or non-target), and (iii) other PFS event, e.g., death, clinical progression, or censoring. Investigator review of disease progression was used as the primary analysis for these investigations. Where progression was determined by investigator review, no further scans were performed, leading to informative censoring of independent review data (approximately 20%). The number of new metastases observed by independent review was considered an under-estimate due to this considerable informative censoring. OS and PFS were compared between treatment arms using two-sided, stratified (geographic region and HER2 status) log-rank tests. The correlation between progression events and OS was investigated by Cox regression, incorporating the event as a time-dependent covariate. New metastasis-free survival, defined as time from randomization to death or progression due to appearance of a new metastasis (whichever occurs earlier), was also analyzed.

Progression due to a new metastasis or increase in the size of a pre-existing lesion occurred in 271 (48.9%) versus 285 (52.0%), and 147 (26.5%) versus 129 (23.5%) of eribulin- and capecitabine-treated patients, respectively. Progression due to other reasons such as death and clinical progression occurred in 136 (24.5%) versus 134 (24.5%) of eribulin- and capecitabine-treated patients, respectively. OS in patients whose disease progressed due to a new metastasis was shorter than in patients whose disease progressed due to an increase in the size of a pre-existing lesion (Table 1). In patients whose disease progressed due to a new metastasis, median OS was 2.6 months longer in patients treated with eribulin compared with those treated with capecitabine (nominal P=0.02), whereas median OS in patients whose disease progressed due to an increase in the size of a pre-existing lesion was similar between arms. In patients whose disease progressed due to other events, median OS was 16.7 versus 15.5 months (HR 0.78; 95% CI 0.59, 1.03; nominal P=0.08).

TABLE 1
Overall survival for patients whose disease progressed due to
a new metastasis or due to an increase in size of pre-existing
lesions

| | Progression due to | | Progression due to | |
|---|---|---|---|---|
| | new metastasis | | pre-existing lesion | |
| | Eribulin | Capecitabine | Eribulin | Capecitabine |
| | n = 271 | n = 285 | n = 147 | N = 129 |
| Median OS, | 15.5 | 12.9 | 17.4 | 17.4 |
| months | (14.2, 17.5) | (11.3, 14.5) | (14.4, 19.7) | (15.3, 20.9) |
| (95% CI) | | | | |

| | | |
|---|---|---|
| HR | 0.81 | 1.13 |
| (95% CI) | (0.68, 0.97) | (0.87, 1.46) |
| P-value | 0.02 | 0.35 |

CI, confidence interval;
OS, overall survival

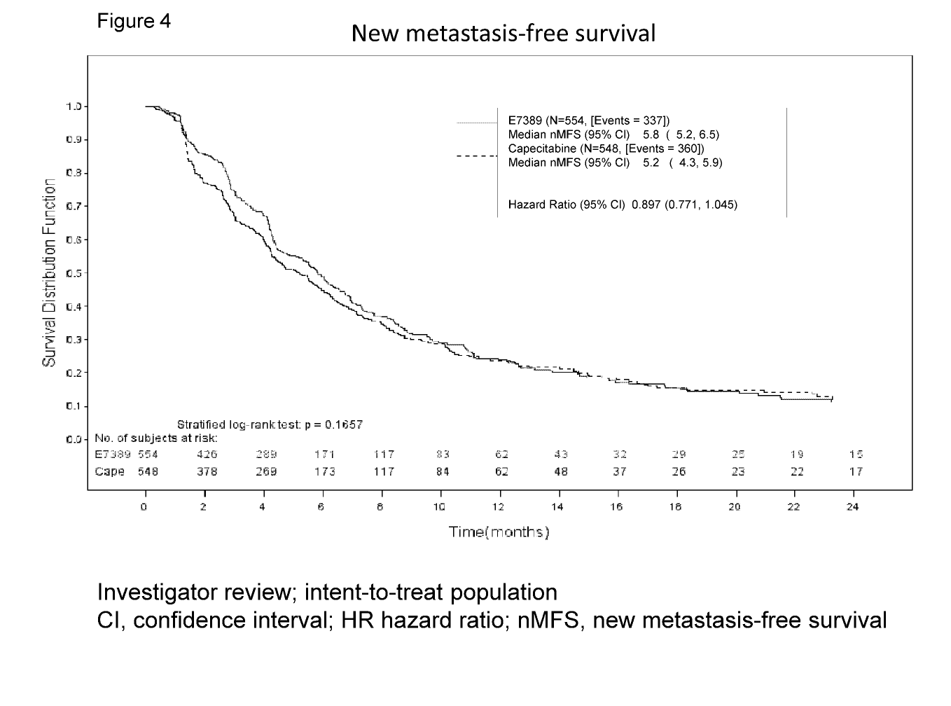

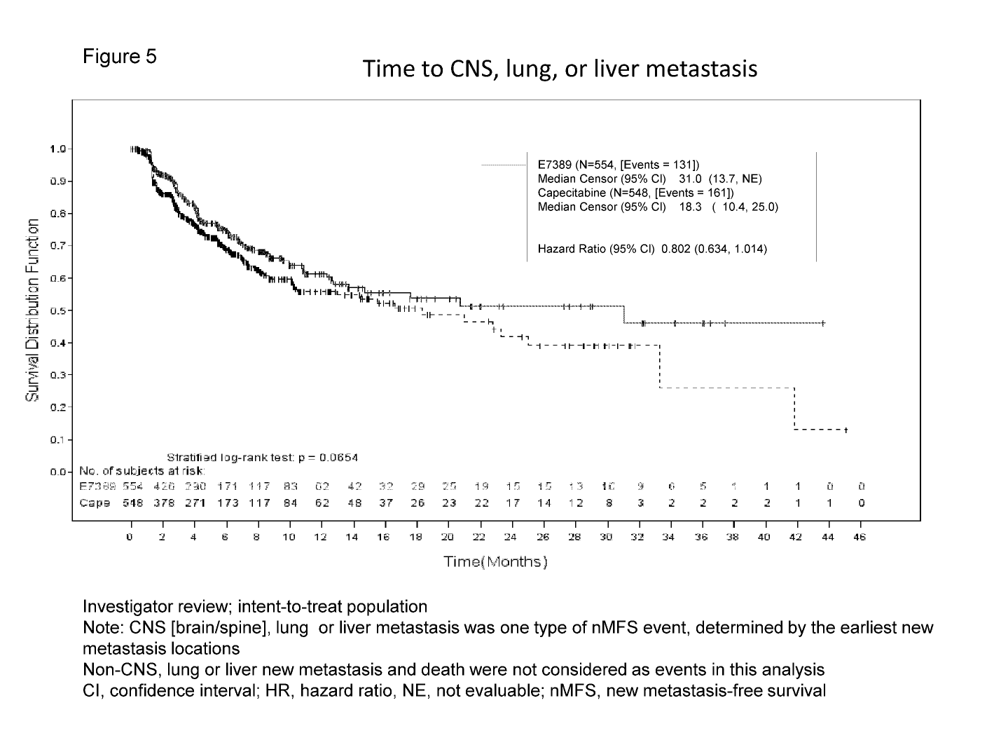

Once patients were deemed to have tumor progression due to a new metastasis, they were at higher risk of death (HR 2.12; 95% CI 1.84, 2.43; Wald nominal P<0.0001; versus tumour progression with no new metastasis, whether stratified by treatment group or not). There was a trend in new metastasis-free survival in favour of eribulin with a median difference of 0.6 months (HR 0.90; 95% CI 0.77, 1.05) (FIG. 4). Data from independent review were largely consistent with investigator review, with a trend in new metastasis-free survival in favour of eribulin (median difference 0.3 months). The incidence of new metastases by site is shown in Table 2. The incidence of new metastasis in the CNS or lungs was lower in eribulin-treated patients compared with patients who received capecitabine. The time to a new metastasis observed in the CNS, lungs, or liver is shown in FIG. 5, with a trend favouring eribulin over capecitabine.

TABLE 2
Summary of new metastasis sites

| | | | | |
|---|---|---|---|---|
| Number of patients with baseline | 554 | (100.0) | 547 | (99.8) |
| tumor scans | | | | |
| Number of patients with any new | 291 | (52.5) | 316 | (57.7) |
| metastasis observed | | | | |
| New metastasis sites* | | | | |
| Liver | 71 | (24.4) | 74 | (23.4) |
| Lung | 59 | (20.3) | 76 | (24.1) |
| Bone | 60 | (20.6) | 62 | (19.6) |
| Lymph nodes | 64 | (22.0) | 55 | (17.4) |
| Skin | 23 | (7.9) | 26 | (8.2) |
| CNS (brain/spine) | 13 | (4.5) | 25 | (7.9) |
| Breast | 12 | (4.1) | 13 | (4.1) |
| Chest wall | 15 | (5.2) | 5 | (1.6) |
| Other | 30 | (10.3) | 53 | (16.8) |

Investigator review; intent-to-treat population
*Percentage for metastasis sites is based on number of patients with any new metastasis observed for each arm. Only the earliest new metastasis observed was recorded. If there were multiple new metastases observed at the same time and all determined as earliest, all new metastasis sites were summarized.

These results suggest that the conventional definition of PFS may not be fully adequate. Patients who are deemed to have tumor progression due to new metastases have a worse prognosis than those whose progression is due to an increase in the size of a pre-existing lesion. Determination of new metastases in the CNS, liver, or lungs is radiologically more straightforward than in the lymph nodes and may also correlate with OS. The discordance between PFS and OS may be due to the heterogeneity between patients having disease progression due to a new metastasis versus patients having disease progression due to increases in the sizes of existing lesions.

### Analysis of Patient Subgroups

Patients were evaluated based on the status of the expression of the following receptors: HER2, ER, and PR. Table 3 provides the number of patients in the study that had cancers characterized by HER2 and hormone receptor status.

TABLE 3
Patient Populations

| | Treated with | Treated with |
|---|---|---|
| Number of Patients | Eribulin (n = 554) | Capecitabine (n = 548) |
| HER2 Positive | n = 86 | n = 83 |
| HER2 Negative | n = 375 | n = 380 |
| Hormone Receptor | n = 279 | n = 305 |
| Positive | | |
| Hormone Receptor | n = 212 | n = 184 |
| Negative | | |
| Triple Negative | n = 150 | n = 134 |

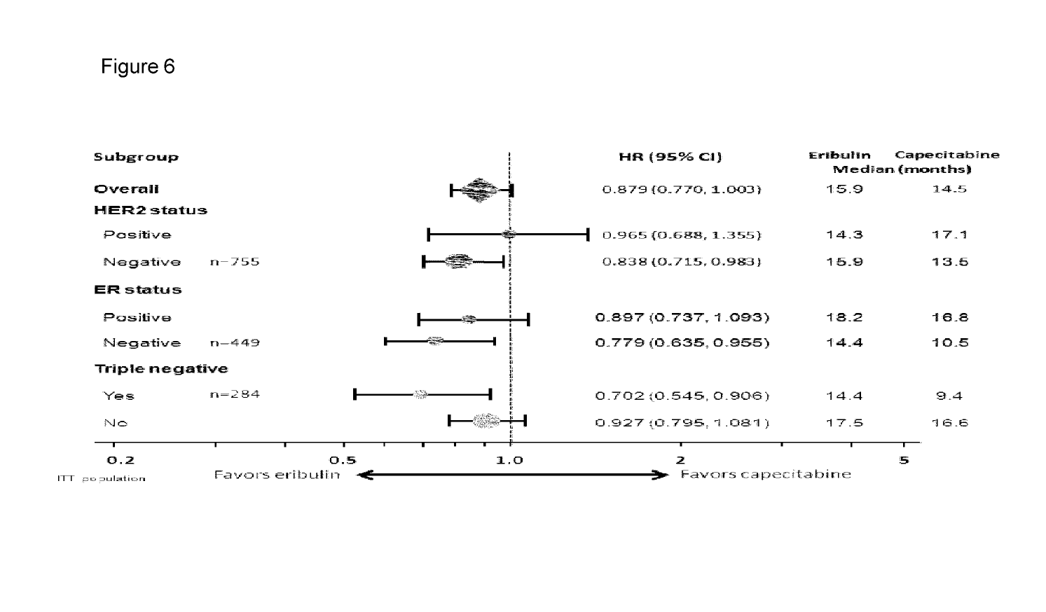

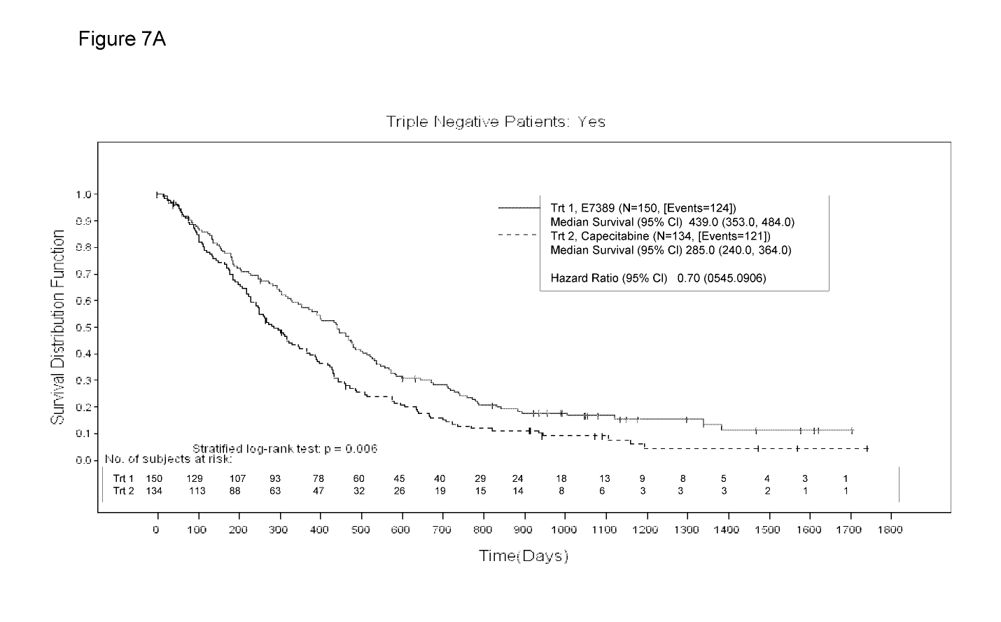

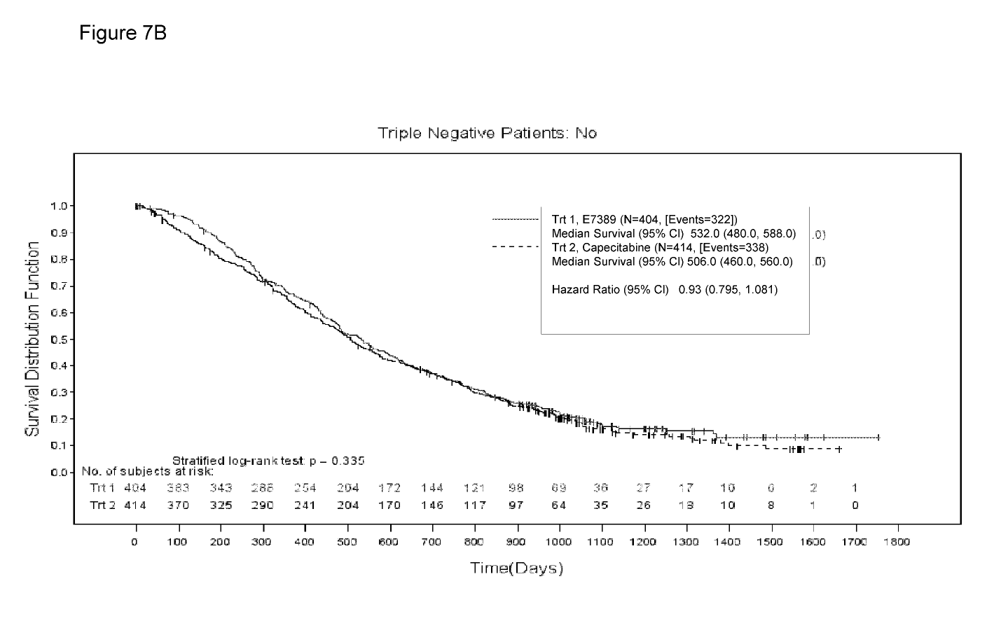

As shown in FIG. 6, HER2 negative patients who were administered eribulin showed increased overall survival as compared to patients who were administered capecitabine. A similar trend was also observed in the case of ER negative patients. However, the most significant improvement due to treatment with eribulin is demonstrated by patients having breast cancer characterized as triple negative (lacking all three receptors). These results show that eribulin may help increase overall survival in specific patient populations based on the expression of HER2, ER, and PR receptors in their breast cancer. The surprising result in triple negative patients is demonstrated even more clearly in FIGS. 7A and 7B.

### Example 2

### Clinical Studies of Eribulin: Phase II Clinical Trial for use of Eribulin as a First-Line Treatment for HER2 Negative Breast Cancer (Part 1)

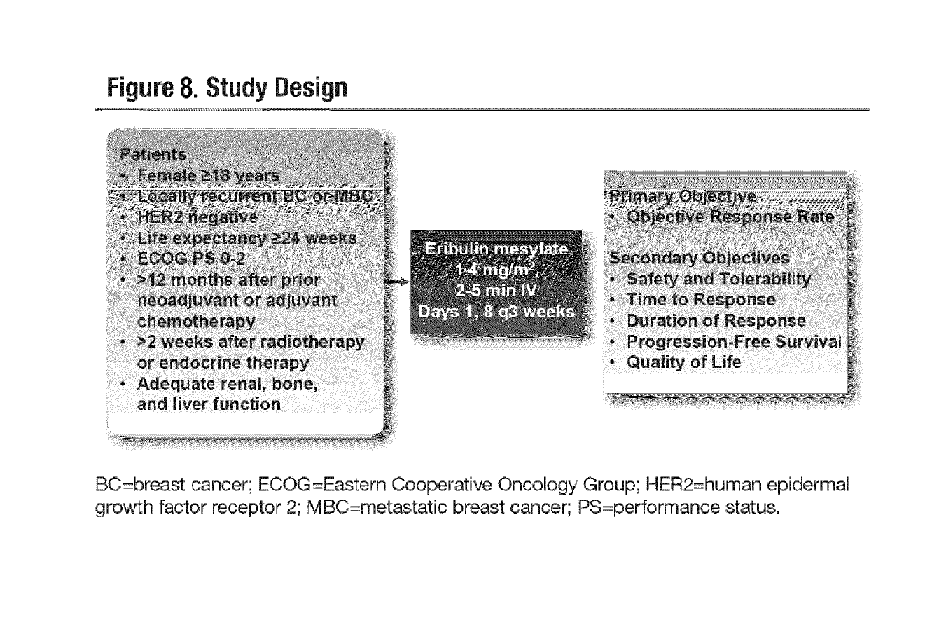

A multi-center, single-arm, phase II clinical trial of eribulin mesylate was performed to evaluate the objective response rate (ORR) (according to RECIST v1.1) to first-line treatment with single-agent eribulin mesylate in subjects with locally recurrent or metastatic HER2 negative breast cancer. Secondary objectives included the safety and tolerability of eribulin mesylate, time to first response, duration of response (DOR), and progression free survival (PFS). The study design, patient eligibility, and study parameters are shown in FIG. 8.

Patients were excluded from the study if they had inflammatory breast cancer or had received prior chemotherapy, biologic therapy, or investigational therapy for locally recurrent or metastatic breast cancer (patients who received prior endocrine therapy were permitted).

HER2 status was determined by either fluorescence in situ hybridization (FISH) or 0 or 1+ by immunohistochemical (IHC) staining. Subjects with an HER2:FISH ratio of 1.8:2.2 were eligible, consistent with ASCO/CAP guidelines (Wolff et al., J. Clin. Oncol. 25:118-145, 2007).

60 patients were screened for participation in the study and 48 received at least 1 dose of eribulin. Twenty-six subjects (54.2%) received all 6 planned cycles of eribulin. The median number of cycles received per patient was 6, with a range of 1 to 17. A total of 38 (79.2%) patients had previously received breast anticancer therapy and 35 (92.1%) had received neoadjuvant and/or adjuvant therapy. Anthracyclines were previously given to 25 (52.1%) patients and taxanes were previously given to 23 (47.9%) patients. 35 of the patients (72.9%) had cancers characterized ad HER2−/ER+. 13 (27.1%) had cancers characterized as HER2−/ER−. 10 patients (20.8%) had breast cancer characterized as triple negative. 30 patients (62%) had received prior therapy with taxane or anthracycline. 18 patients (37.5%) hand not previously been treated with taxane or anthracycline.

### Efficacy Outcomes

Of the 48 patients enrolled, 47 had at least 1 post-baseline assessment. As seen in Table 4, the objective response rate (ORR) was 27.1% (13/48). Subgroups with either HER2−/ER+ or triple negative (ER−/PR−/HER2−) status were analyzed.

TABLE 4
Tumor Response to Eribulin

| | | | Triple Negative |
|---|---|---|---|
| | All, | ER+, | (ER−/PR−/HER2−), |
| Response category | n (%) N = 48 | n (%) N = 35 | n (%) N = 10 |
| Objective response rate (ORR) | 13 (27.1) | 10 (28.6) | 3 (30.0) |
| 95% CI | (15.28, 41.85) | (14.64, 46.30) | (6.67, 65.25) |
| Complete response (CR) | 0 | 0 | 0 |
| Partial response (PR) | 13 (27.1) | 10 (28.6) | 3 (30.0) |
| Stable disease (SD) | 23 (47.9) | 19 (54.3) | 3 (30.0) |
| Progressive disease (PD) | 11 (22.9) | 6 (17.1) | 3 (30.0) |
| Not evaluable/unknown | 1 (2.1) | 0 | 1 (10.0) |
| Clinical benefit rate | 22 (45.8) | 19 (54.3) | 3 (30.0) |
| (CR + PR + durable SD) | | | |
| 95% CI | (31.37, 60.83) | (36.65, 71.17) | (6.67, 65.25) |
| Disease control rate | 36 (75.0) | 29 (82.9) | 6 (60.0) |
| (CR + PR + SD) | | | |
| 95% CI | (60.40, 86.36) | (66.35, 93.44) | (26.24, 87.84) |

\grave{ }Note:
3 patients were ER−/PR+ with no objective response (1 SD, 2 PD)

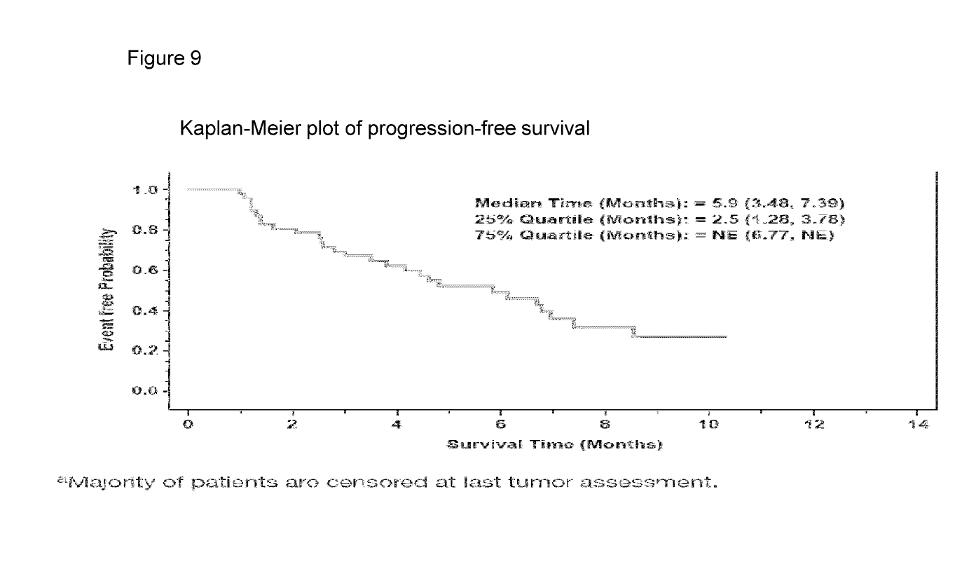

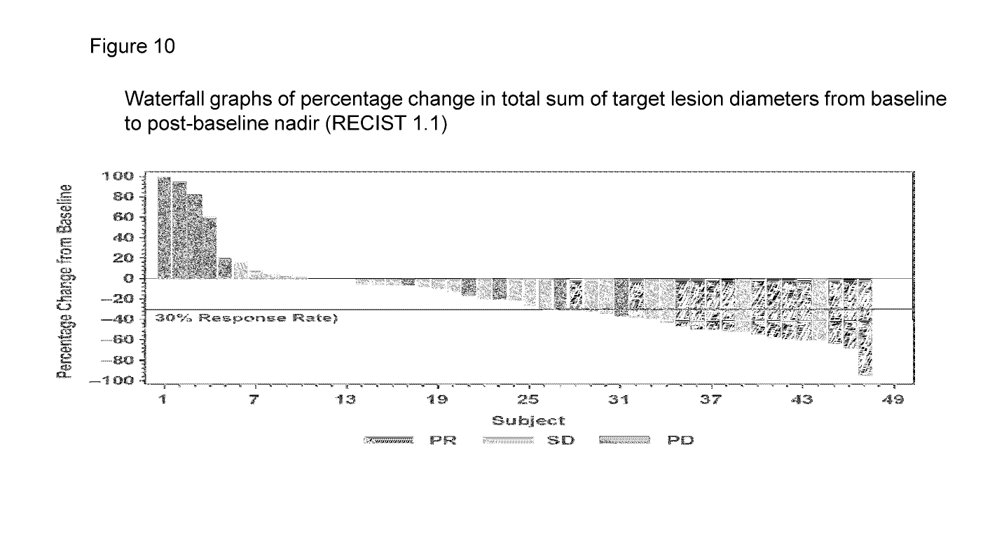

For the 13 partial responders, the median time to first response was 1.4 months (95% CI, 1.31-2.69 months) and the median duration of objective response was 7.4 months (95% CI, 3.29 to NE*). For all patients treated, the median progression-free survival was 5.9 months (95% CI, 3.48-7.39) (Table 5, and FIG. 9). As shown in FIG. 10, the majority of patients experienced a decrease in the sum of target lesion diameters from baseline to post-baseline nadir.

TABLE 5
Secondary Efficacy Outcomes

| | | | | | Triple Negative (ER−/ | |
|---|---|---|---|---|---|---|
| | All Subjects | | ER+ Subjects | | PR−/HER2−) Subjects | |
| Response | | Median months | | Median Months | | Median months |
| category | N | (95% CI) | N | (95% CI) | N | (95% CI) |
| PFS (months) | 48 | 5.9 (3.48, 7.39) | 35 | 6.7 (4.14, 8.54) | 10 | 4.7 (1.05, NE) |
| TTR (months) | 13 | 1.4 (1.31, 2.69) | 10 | 1.4 (1.22, 2.66) | 3 | 2.9 (1.15, 5.59) |
| DOR (months) | 13 | 7.4 (3.29, NE) | 10 | 7.4 (3.29, NE) | 3 | NE (4.73, NE) |

\hat{ }NE = Not evaluable

These results show that eribulin has antitumor activity in HER2−/ER+ and triple negative (HER2−/ER−/PR−) metastatic/recurrent breast cancer, thus supporting its use as a first-line treatment for metastatic breast cancer.

### Example 3

### Clinical Studies of Eribulin: Phase II Clinical Trial for use of Eribulin as a First-Line Treatment for HER2 Negative Breast Cancer (Part 2)

This example provides additional data obtained from the study described above in Example 2. The baseline demographics and characteristics of patients in the study are as set forth below in Table 6.

TABLE 6
Baseline Demographics and Characteristics

| | Eribulin-treated patients |
|---|---|
| Characteristic | N = 56 |
| Age, years | |
| Mean (SD) | 57.0 (10.8) |
| Range | 31-85 |
| Race, n (%) | |
| Caucasian | 42 (75.0) |
| Black/African American | 12 (21.4) |
| Asian | 1 (1.8) |
| Other | 1 (1.8) |
| ECOG performance status, n (%) | |
| 0 | 32 (57.1) |
| 1 | 21 (37.5) |
| 2 | 3 (5.4) |
| Breast cancer stage, n (%) | |
| Stage IV | 56 (100) |
| Time from original diagnosis to | |
| metastatic breast cancer, n (%) | |
| <3 months | 17 (30.4) |
| ≧3 months | 39 (69.6) |
| ER/PR status, n (%) | |
| ER+ or PR+ | 44 (78.6) |
| ER− and PR− | 12 (21.4) |
| Triple negative (ER−/PR−/HER2−), n (%) | 12 (21.4) |
| Site of metastases, n (%) | |
| Visceral | 39 (69.6) |
| Liver | 25 (44.6) |
| Lung | 18 (32.1) |
| Nonvisceral | 17 (30.4) |
| Preexisting neuropathy, n (%) | 9 (16.1) |
| Prior anticancer therapy (breast), n (%) | 42 (75.0) |
| Anthracycline therapy (any setting) | 27 (48.2) |
| Neo/adjuvant therapy | 38 (67.9) |
| Taxaries | 25 (44.6) |
| Time since neo/adjuvant therapy, n (%) | |
| <2 years | 10 (17.9) |
| ≧2 years | 28 (50.0) |
| No neo/adjuvant therapy | 18 (32.1) |

ECOG = Eastern Cooperative Oncology Group;
ER = estrogen raceptor;
PR = progesterone receptor;
SD = standard deviation.

As of this update, of the 68 screened patients, 56 received at least 1 dose of eribulin (12 screen failures were due to: not meeting inclusion/exclusion criteria [n=7], adverse event [n=1], withdrawal of consent [n=1], and other [n=3]. 32 patients (57%) received all 6 planned cycles of eribulin. The median number of cycles delivered was 7 (range, 1-43). A total of 42 (75%) patients had previously received breast cancer therapy; of these 42 patients, 38 (90.5%) received neoadjuvant or adjuvant therapy; taxanes (as neoadjuvant/adjuvant therapy) were previously given to 25 patients. 27 patients had received prior anthracycline therapy (in any setting).

### Efficacy Outcomes

The ORR was 28.6% (16/56; 95% CI, 17.3-42.2) (Table 7). The ORR among patients who had received new/adjuvant treatment with anthracyclines and/or taxanes (A/T) was 27.3% (9/33) and the clinical benefit rate (CBR) was 45.5% (15/33) and were similar to the overall population. There was no difference in median PFS in patients who had received prior A/T (5.9 months) compared with those who did not (5.7 months). Subgroups with either estrogen receptor positive (ER+) or triple negative (ER−/PR−/HER2−) status were analyzed and results are reported below (Table 7). Patients within ER+ subgroup performed better (ORR 34.1%, disease control rate 85.4%, PFS 7.4 months); however, the number of patients was small.

TABLE 7
Best Tumor Responses

| | | | Triple negative |
|---|---|---|---|
| | ALL | ER+ | (ER−/PR−/HER2−) |
| Response category, n (%) | N = 56 | n = 41 | n = 12 |
| Objective response rate | 16 (28.6) | 14 (34.1) | 2 (16.1) |
| (ORR) | | | |
| 95% CI | 17.3-42.2 | 20.1-50.6 | 2.1-43.4 |
| Complete response (CR) | 0 | 0 | 0 |
| Partial response (PR) | 16 (28.6) | 14 (34.1) | 2 (16.7) |
| Stable disease (SD) | 26 (46.4) | 21 (51.2) | 4 (33.3) |
| Progressive disease (PD) | 11 (19.6) | 5 (12.2) | 5 (41.7) |
| Not evaluable/unknown | 3 (5.4) | 1 (2.4) | 1 (8.3) |
| Clinical benefit rate | 29 (51.8) | 26 (63.4) | 3 (25.0) |
| (CR + PR + | | | |
| ≧6 months SD) | | | |
| 95% CI | 38.0-65.3 | 46.9-77.9 | 5.5-57.2 |
| Disease control rate | 42 (75.0) | 35 (85.4) | 6 (50.0) |
| (CR + PR + SD) | | | |
| 95% CI | 61.6-85.6 | 70.8-94.4 | 21.1-78.9 |

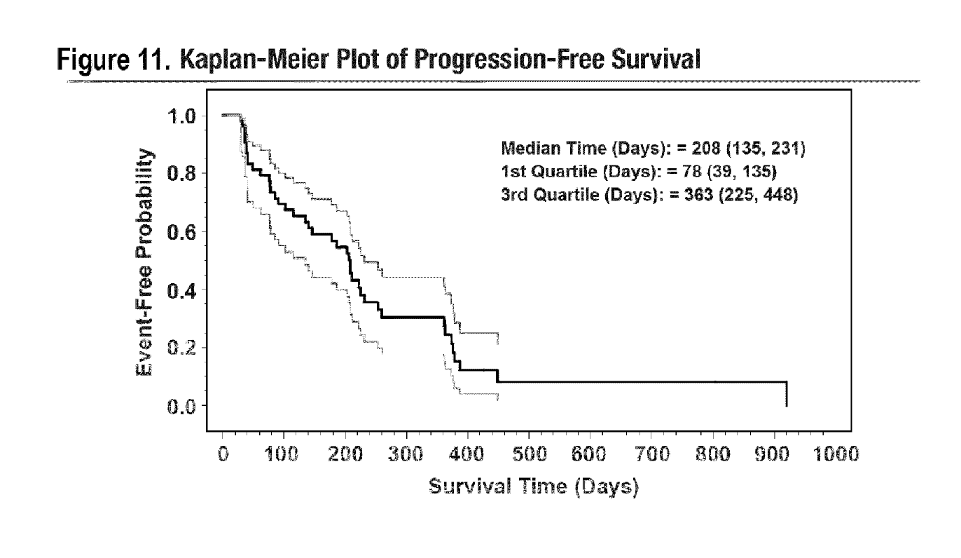

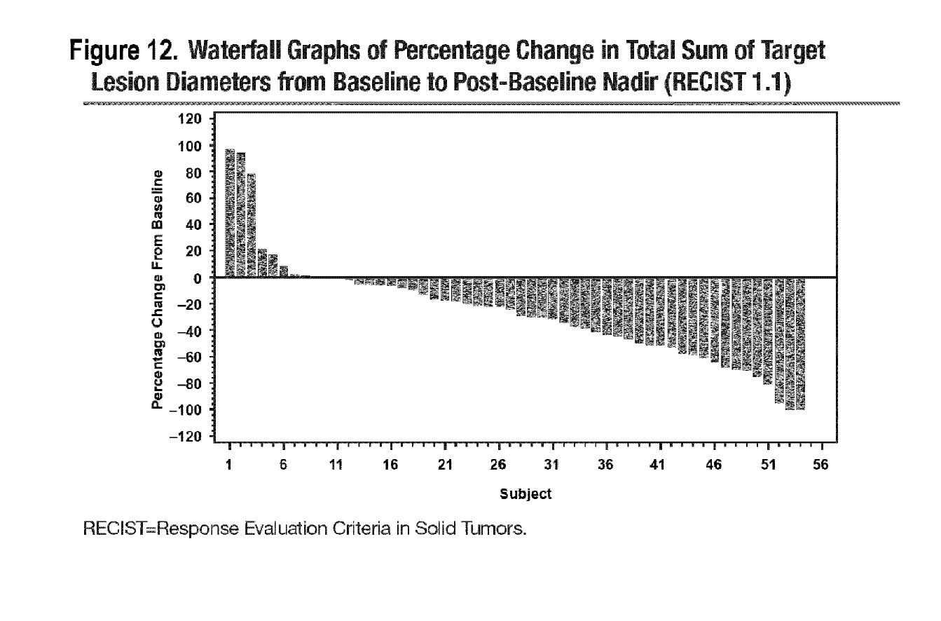

For the 16 patients with partial response, median time to first response was 1.4 months (95% CI, 1.2-2.7) (Table 8), and median DOR was 5.8 months (95% CI, 4.7-10.6) (Table 8). For all patients treated, the PFS was 6.8 months (95% CI, 4.4-4.7) (FIG. 11; Table 8). The majority of patients experienced a decrease in the sum of target lesion diameters from baseline to postbaseline nadir (FIG. 12).

TABLE 8
Secondary Efficacy Outcomes

| | ALL | | ER+ | | ER−/PR−/HER2− | |
|---|---|---|---|---|---|---|
| | | Median | | Median | | Median |
| Response | | months | | months | | months |
| category | N | (95% CI) | n | (95% CI) | n | (95% CI) |
| PFS | 56 | 6.8 (4.4-7.6) | 41 | 7.4 (6.1-11.9) | 12 | 3.4 (1.2-6.8) |
| TTR | 16 | 1.4 (1.2-2.7) | 14 | 2.0 (1.3-2.7) | 2 | 1.1* |
| DOR | 16 | 5.8 (4.7-10.6) | 14 | 7.4 (4.6-27.5) | 2 | 5.2* |

DOR = duration of response; PFS = progression-free survival; TTR = time to first response.
*CI not available (n = 2).

### Safety Outcomes

The overall incidence of the following markedly abnormal lab values was reported: low hemoglobin: 14.5% (8/55), low leukocytes: 57.4% (31/54), low lymphocytes: 18.2% (8/44), low neutrophils: 77.4% (41/53), low platelets: 2% (1/51) (Table 9). Throughout the study, 5.8% of patients (3/52) had an abnormal but not clinically significant finding.

TABLE 9
Most Common Treatment-Related AEs (Incidence >25%)

| | All grades, n (%) | Grade 3/4, n (%) |
|---|---|---|
| AE | N = 56 | N = 56 |
| Alopecia | 47 (83.9) | NA |
| Neutropenia | 40 (71.4) | 28 (50.0) |
| Fatigue | 34 (60.7) | 1 (1.8) |
| Peripheral neuropathy^{a} | 32 (57.1) | 11 (19.6) |
| Nausea | 27 (48.2) | 0 |
| Anemia | 20 (35.7) | 2 (3.6) |
| Leukopenia | 19 (33.9) | 12 (21.4) |
| Constipation | 15 (26.8) | 0 |
| Diarrhea | 14 (25.0) | 0 |

AE = adverse event;
NA = not appiicable.
Note:
Febrile neutropenia (grade 3/4) was reported for 4 patients.
^{a}Peripheral neuropathy includes the following preferred terms: Neuropathy Peripheral, Neuropathy, Peripheral Motor Neuropathy Polyneuropathy, Peripheral Sensory Neuropathy, Peripheral Sensorimotor Neuropathy, Demyelinating Polyneuropathy, and Paresthesia.
There were no grade 5 related events.

Overall, 36 (64.3%) patients experienced a treatment-related AE with a Common Terminology Criteria for Adverse Events (CTCAE) grade ¾. Treatment-related SAEs occurred in 5 (8.9%) patients. Febrile neutropenia occurred in 3 (5.4%) patients, and leucopenia in 1 (1.8%) patient. Treatment-related AEs led to dose adjustment (interruption/delay, reduction, or discontinuation) in 30 (53.6%) patients: 20 (35.7%) patients had their dose reduced; 20 (35.7%) had their dose interrupted/delayed; 6 (10.7%) patients discontinued the study due to an AE. Peripheral neuropathy was responsible for 5 of the 6 events resulting in discontinuations. The median time to first occurrence of peripheral neuropathy was 4 months. The duration of grade ¾ peripheral neuropathy was short (median 2.3 months) due to proper dose modification. The remaining patient experienced a prolonged QT interval requiring drug withdrawal. The median relative dose intensity in the first 6 cycles was 99% (range, 47.6-101.3). Growth factors were administered to 22 (39.3%) patients, with a median start of 2.6 weeks (18 days) from the first dose of study drug.

Of the 48 patients enrolled, 47 had at least 1 post-baseline assessment. As seen in Table 4, the objective response rate (ORR) was 27.1% (13/48). Subgroups with either HER2−/ER+ or triple negative (ER−/PR−/HER2−) status were analyzed.

### DISCUSSION AND CONCLUSIONS

The results of this first-line study show that eribulin has antitumor activity in ER+/HER2− and triple negative (ER−/PR−/HER2−) metastatic/recurrent breast cancer with an acceptable safety profile. Safety was consistent with known profile for eribulin. Alopecia, neutropenia, fatigue, and peripheral neuropathy were the most commonly observed treatment-related AEs (all occurring in >50% of patients). The most common grade ¾ AE was neutropenia, occurring in 50% of patients. Febrile neutropenia (grade ¾) was reported for 4 patients. Six patients discontinued due to an AR

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated as being incorporated by reference in their entirety.

Use of singular forms herein, such as “a” and “the,” does not exclude indication of the corresponding plural form, unless the context indicates to the contrary. Similarly, use of plural terms does not exclude indication of a corresponding singular form. Other embodiments are within the scope of the following claims.

What is claimed is:

1. A method of treating breast cancer in a subject selected for having (i) HER2-negative, (ii) estrogen receptor (ER)-negative, or (iii) HER2-negative, ER-negative, and progesterone receptor (PR)-negative (triple negative) breast cancer, the method comprising administering to the subject eribulin or a pharmaceutically acceptable salt thereof.

2. The method of claim 1, wherein the breast cancer is locally advanced breast cancer.

3. The method of claim 1, wherein the breast cancer is metastatic breast cancer.

4. The method of claim 1, wherein the subject has undergone no prior breast cancer treatment regimen.

5. The method of claim 1, wherein the subject has undergone one prior breast cancer treatment regimen.

6. The method of claim 1, wherein the subject has undergone two or more prior breast cancer treatment regimens.

7. The method of claim 5, wherein the prior breast cancer treatment regimen(s) comprise chemotherapy or biologic therapy.

8. The method of claim 5, wherein the subject has undergone a prior breast cancer treatment regimen involving administration of one or more of an antibody, a hormonal agent, capecitabine, an anthracycline, and a taxane.

9. The method of claim 8, wherein the anthracycline is selected from the group consisting of doxorubicin, epirubicin, daunorubicin, and idarubicin.

10. The method of claim 8, wherein the taxane is selected from the group consisting of paclitaxel and docetaxel.

11. The method of claim 8, wherein the antibody is trastuzumab.

12. The method of claim 1, wherein the subject has not previously been treated with an anthracycline or a taxane.

13. The method of claim 1, wherein the subject has HER2-negative breast cancer.

14. The method of claim 1, wherein the subject has ER-negative breast cancer.

15. The method of claim 1, wherein the subject has HER2-negative, ER-negative, and PR-negative (triple negative) breast cancer.

16. The method of claim 1, wherein the pharmaceutically acceptable salt of eribulin is eribulin mesylate.

17. The method of claim 1, wherein the method includes a step of selecting a subject having (i) HER2-negative, (ii) estrogen receptor (ER)-negative, or (iii) HER2-negative, ER-negative, and progesterone receptor (PR)-negative (triple negative) breast cancer for treatment.

18. The method of claim 1, wherein the method includes a step of testing a breast cancer sample from the subject for ER, PR, and/or HER2 status.

19. The method of claim 1, wherein the eribulin or pharmaceutically acceptable salt thereof is administered for 2-5 minutes intravenously on days 1 and 8 of a 21 day cycle, optionally at a dose of 1.4 mg/m².

20. The method of claim 1, wherein the subject is a human.

21. The method of claim 1, further comprising:
(a) selection of eribulin or a pharmaceutically acceptable salt thereof to treat said subject, instead of capecitabine, or
(b) increasing 1-year overall survival by treatment of said subject with eribulin or a pharmaceutically acceptable salt thereof, relative to capecitabine,
based on detection of the breast cancer of said subject as being (i) HER2-negative, (ii) estrogen receptor (ER)-negative, or (iii) HER2-negative, ER-negative, and progesterone receptor (PR)-negative (triple negative).

22-29. (canceled)
